(19) United States
(12) Patent Application Publication
(10) Pub. No.: US 2024/0130636 A1
(43) Pub. Date: Apr. 25, 2024

(54) APPARATUS AND METHOD FOR MOTION CAPTURE

(71) Applicant: YANA HEALTH SYSTEMS LTD. (DBA Yana Motion Lab), Calgary (CA)

(72) Inventors: Douglas Ross Hamilton, Calgary (CA); Joseph Christie Paul Kitson, Calgary (CA); Patrick Crawford Hamilton, Calgary (CA)

(21) Appl. No.: 18/278,499

(22) PCT Filed: Feb. 24, 2022
(86) PCT No.: PCT/CA2022/050264

§ 371 Date: Aug. 22, 2023

Related U.S. Application Data
(60) Provisional application No. 63/153,251, filed on Feb. 24, 2021.

(51) Int. Cl. A61B 5/11; A61B 5/00

(52) U.S. Cl. CPC A61B 5/1126; A61B 5/4023; A61B 5/742

(57) ABSTRACT

A system for analyzing data from motion capture measurements has a sensor to measure motion capture measurements, a module configured to receive sensor data from the sensors, and a processor configured to analyze to the sensor data received from the sensors and create a report of such analysis. A computer-implemented method of analyzing data from motion capture measurements includes performing motion capture measurements on a subject, receiving motion capture data from the motion capture measurements, processing the motion capture data and the background data on a computer using an algorithm, including comparing the motion capture data and background data to one or more comparator waveforms, generating a report based on the processing analysis, and communicating the results of the report.

### BENEFIT OF EARLIER APPLICATIONS

This application claims priority from U.S. provisional application 63/153,251, filed Feb. 24, 2021.

### TECHNICAL FIELD

The present invention relates to motion capture in general, and apparatus, systems, and computer-implemented methods for the collection and analysis of motion capture data in particular.

### SUMMARY OF INVENTION

In accordance with a broad aspect of the present invention, there is provided a system for analyzing data from motion capture measurements, comprising: a sensor to measure motion capture measurements; a module configured to receive sensor data from the sensors; and a processor configured to analyze to the sensor data received from the sensors and create a report of such analysis.

In accordance with another broad aspect of the present invention, there is provided a computer-implemented method of analyzing data from motion capture measurements, comprising: performing motion capture measurements on a subject; receiving motion capture data from the motion capture measurements; processing the motion capture data and the background data on a computer using an algorithm, including comparing the motion capture data and background data to one or more comparator waveforms; generating a report based on the processing analysis; and displaying the results of the report.

It is to be understood that other aspects of the present invention will become readily apparent to those skilled in the art from the following detailed description, wherein various embodiments of the invention are shown and described by way of illustration. As will be realized, the invention is capable of other and different embodiments and its several details are capable of modification in various other respects, all within the present invention. Furthermore, the various embodiments described may be combined, mutatis mutandis, with other embodiments described herein. Accordingly, the drawings and detailed description are to be regarded as illustrative in nature and not as restrictive.

### BACKGROUND

In the area of sports, medicine, and overall human wellness, different areas of expertise may be used to ensure that a subject or patient has the best opportunity to achieve their full motion potential. The ability to perform motion and locomotion is important to all humans in the environments in which they find themselves (e.g., sports, military, workplace, leisure, etc.), and the physical or mental state they have acquired or are developing (healthy or injured, having disease, illness, etc.). Effective motion and locomotion are essential for homeostasis or competitive success and sometimes require, or may benefit from, physical remediation or therapy and possibly supplementation with other devices to achieve this (cane, scooter, splint, etc.). The areas of expertise that use motion analysis to evaluate locomotion can include athletic training, physical therapy, occupational therapy, physiotherapy, medical therapy, legal injury and recovery evaluation, military training and evaluation, and insurance actuarial injury and illness classification, to name a few. Currently, each of the practices that involve the above professions or entities may assess motion, locomotion and/or performance of a subject or patient in many different ways. Some of these methods are very time consuming and involve fastidious attention to measurement procedures to achieve high accuracy. In some cases, such methods may rapidly provide qualitative evaluations (e.g., naked eye or manual goniometer); however, human error may be more prevalent, and such results may not be repeatable, consistent, and/or reliable.

### BRIEF DESCRIPTION OF THE DRAWINGS

Referring to the drawings, several aspects of the present invention are illustrated by way of example, and not by way of limitation, in detail in the drawings, wherein:

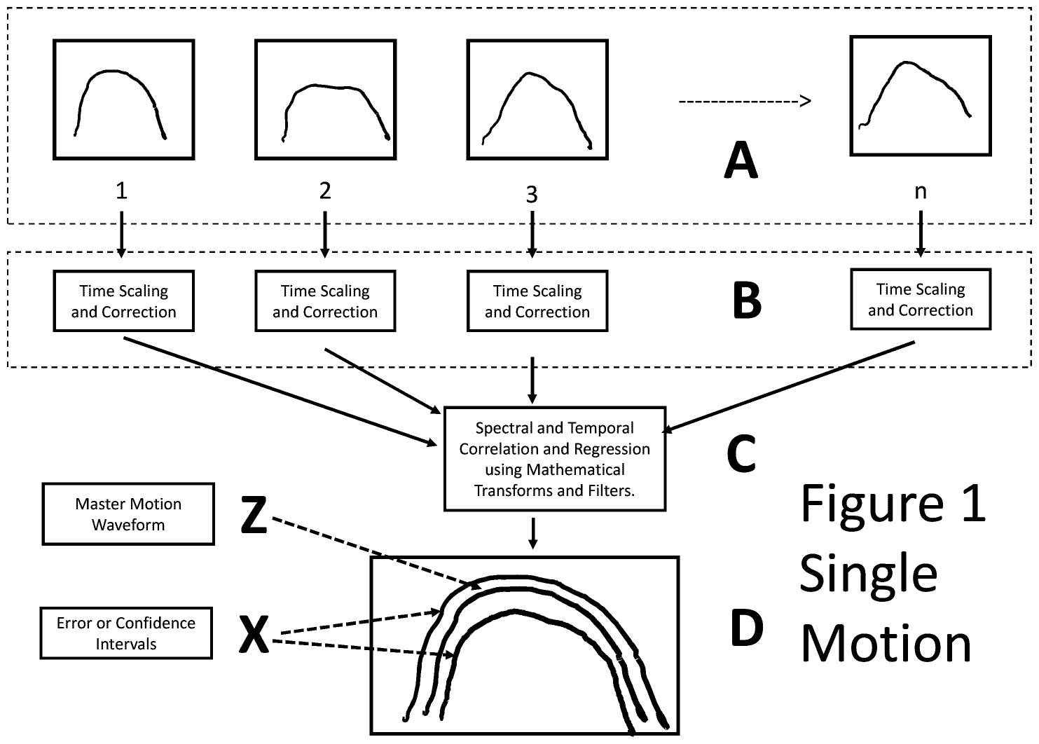

(a) FIG. 1 is a schematic of a method of analyzing data from motion capture measurements, according to one embodiment, applied to a single motion;

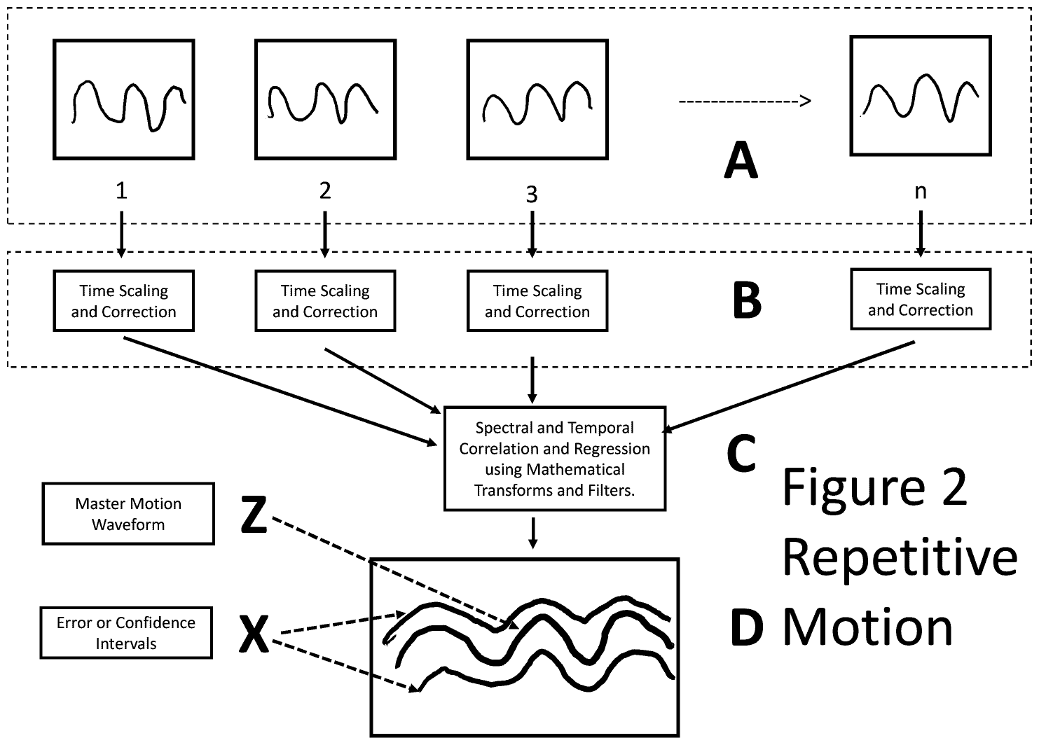

(b) FIG. 2 is a schematic of a method of analyzing data from motion capture measurements, according to another embodiment, applied to a repetitive motion;

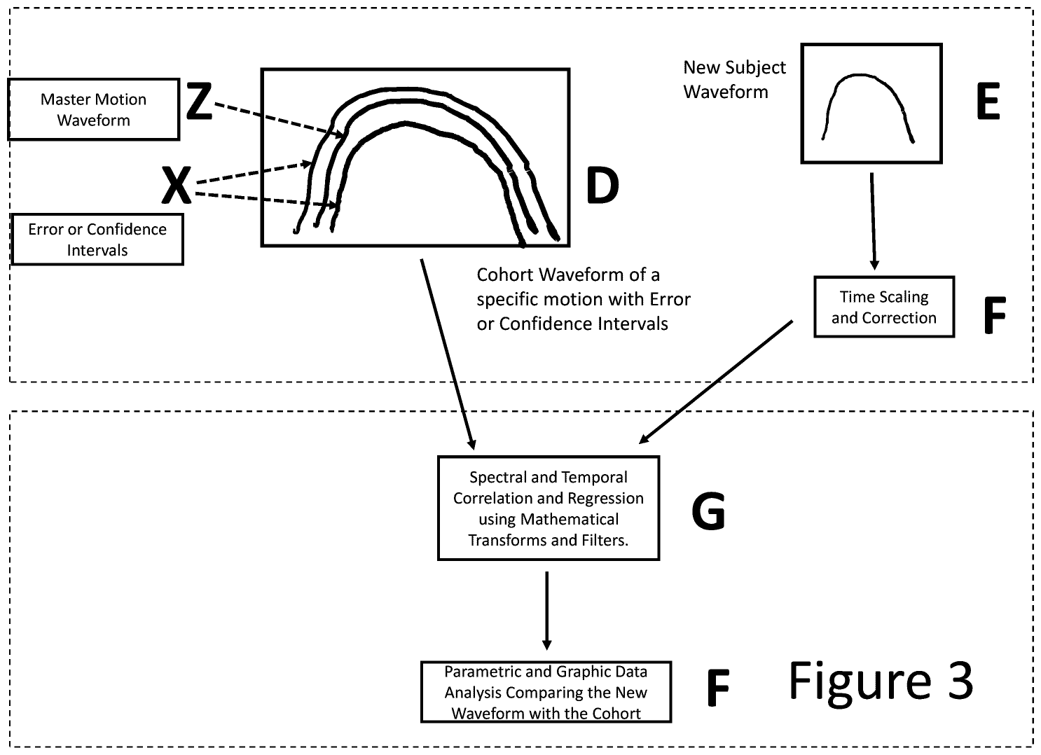

(c) FIG. 3 is a schematic of a method of analyzing data from motion capture measurements, according to another embodiment, including a comparison of a subject's data with comparator data;

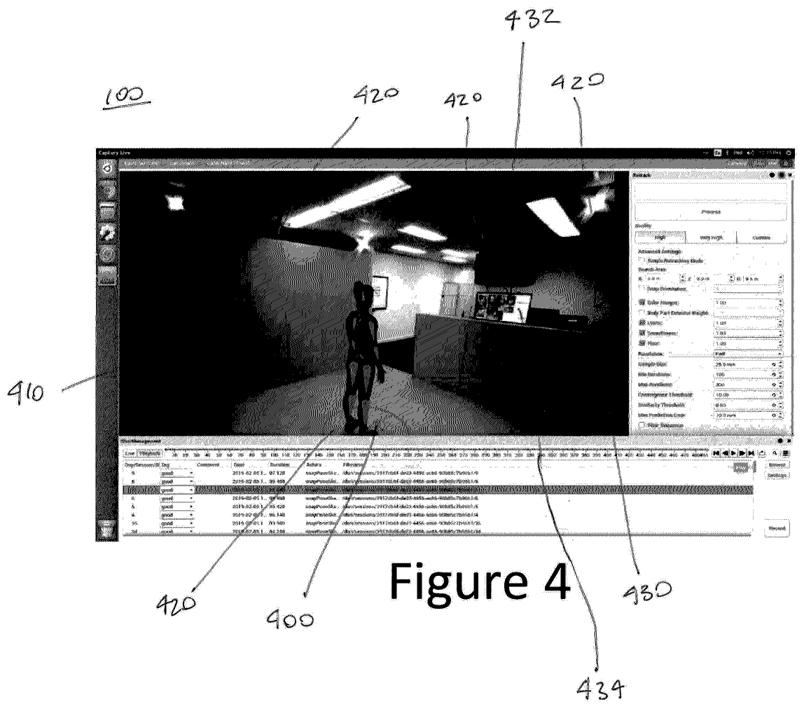

(d) FIG. 4 is a schematic representation of a system of analyzing data from motion capture measurements, according to one embodiment, including a studio;

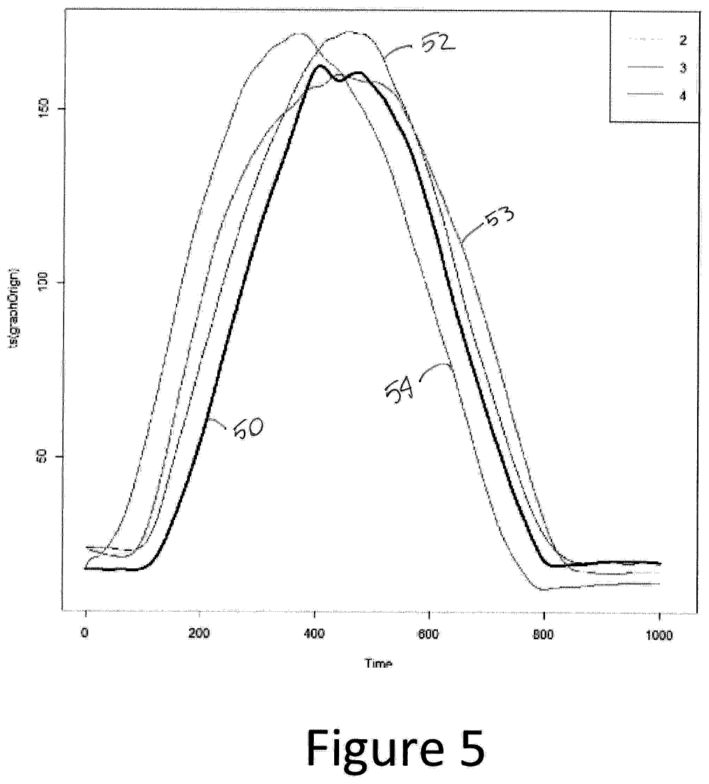

(e) FIG. 5 is a graph illustrating a waveform of sample data of a shoulder abduction waveform, according to one embodiment of the present method and system;

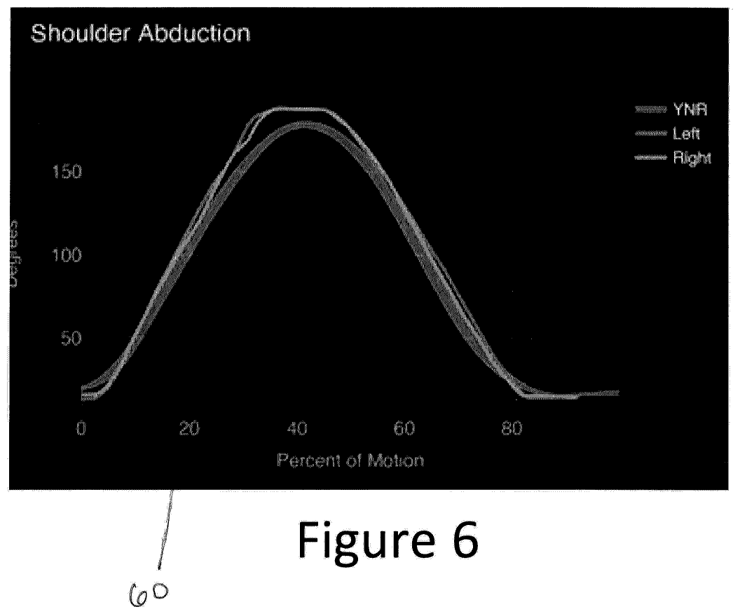

(f) FIG. 6 is a graph illustrating a waveform of sample data of a shoulder abduction waveform, according to another embodiment of the present method and system; and

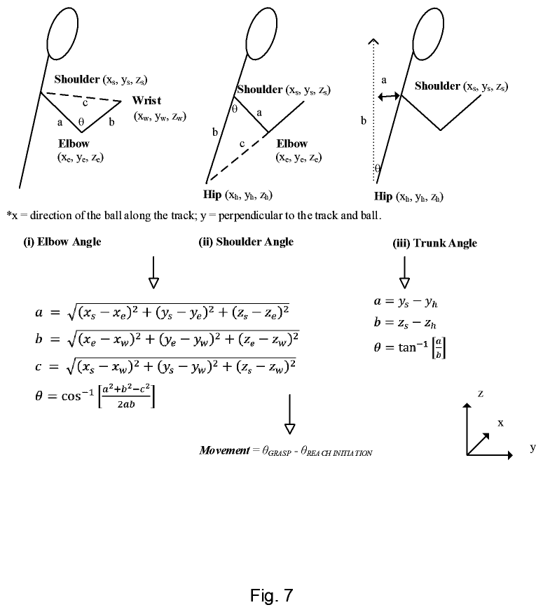

(g) FIG. 7 is a diagram illustrating mathematical principles that may be implemented according to one embodiment of the present method and system.

### DETAILED DESCRIPTION OF EMBODIMENTS

The detailed description set forth below in connection with the appended drawings is intended as a description of various embodiments of the present invention and is not intended to represent the only embodiments contemplated by the inventor. The detailed description includes specific details for the purpose of providing a comprehensive understanding of the present invention. However, it will be apparent to those skilled in the art that the present invention may be practiced without these specific details.

The present apparatus and methods, in one aspect, may be useful in is in the therapeutic, diagnostic, and prognostic evaluation of human movement in clinical, medical, and athletic populations. Movement or motion data of a subject can be captured and digitized with motion capture technologies (marker-based methods, markerless methods, lidar, etc.), which may use one or more sensors to capture objective measurements and data of kinematics (motion) and kinetics (forces). Examples of such apparatus and methods include Scientific Analytics Incorporation (SAI) Dynamic Research Institute (DART) motion analysis, Captury, lidar, etc. Such methods and apparatus may generate three-dimensional data, which may be represented as a matrix (e.g., in Biovision Hierarchy (BHV), motion file (.MOT), and/or other formats) to describe the position and/or velocity of various body segments of a subject, either at a particular time or over a period of time.

Such methods and processes may be, in part, be carried out by workers, who may act as observers, (e.g., clinicians, technicians, physiotherapists, subject matter experts, etc.) to decide what aspect of human motion may be the focus of examination, and the same and/or other workers who may interpret the results of data produced by such methods, such interpretation being informed by the workers' areas of expertise. Examination procedures, diagnosis and prognosis have considerable variability due to subjectivity in collecting data and interpreting same.

As such, motion capture is sometimes regarded as an objective methodological investigational technique rather than a diagnostic procedure. There is a demand for a full-body motion-capture method that does not rely on markers positioned on the body, that can objectively capture data and support the interpretation of same. Such a method may reduce error by reducing subjectivity and/or human intervention, and increasing consistency and/or reliability of data acquisition and interpretation.

The ability of a subject to maintain centre of mass over the body's base of support may be characterized as a complex integration of sensorimotor control systems that, in healthy individuals, act in concert, making postural adjustments in various environmental situations. Neural signals from the vestibular apparatus of the inner ear may provide information for linear and/or angular acceleration of the head. Visual information may provide spatial data relative to the visible environment, and proprioceptive and pressure information from sensory neurons within the musculoskeletal system may provide information on individual joint orientation and loading. To maintain balance and postural stability, some or all of these signals may be processed centrally in the brain, to facilitate automatic cerebellar adjustments to muscle tone and joint position, to maintain or attempt to maintain a stable centre of mass within a desired position or with an instant velocity that may be further confirmed or informed by visual information. Damage or impairment of any sensory or motor inputs or outputs or the neurological organs which process and react to these sensory signals has the potential to manifest as an external balance instability. Examples of such impairments include:
(a) Pathologies that target the vestibular apparatus such as Meniere's disease present with specific equilibrium disorders;
(b) Cerebral vascular accidents or closed head trauma, which can effect almost any component of the neurological system;
(c) Musculoskeletal injuries that may damage proprioceptive organelles, which can alter balance responses that increase injury risk in athletes requiring optimal movement patterns;

(d) Conditions such as Parkinson's that alter motor planning within the pre-motor cortex of the brain, or concussive injuries that cause an energy crisis within the brain that alters the processing ability of the brain, which may manifest with disorders of balance; and

(e) Metabolic conditions, which can cause balance disorders, such as diabetes with prolonged inadequate glycemic control, which may result in sensory and motor neuropathies that can embarrass the patient's ability to maintain a stable posture.

The previous examples include or can give rise to balance disorders, which may be characterized by different presentation of static and dynamic posture and balance control, which may be unique to, and/or indicative of, the pathology of their illness or injury, or a candidate pathology, illness, or injury for further examination. The accurate measurement of posture and balance stability using markerless motion image capture is useful for the diagnosis, treatment, and prognosis of malady. The means to measure centre of mass positional data in relation to other anatomical landmarks without interfering with the proprioceptive system (unlike with the use of markers) presents an opportunity for a ubiquitous diagnostic and clinical measurement process.

In one embodiment, a system 100 for analyzing data from motion capture measurements is provided. The system may include a studio 400, which may be characterized as a set. The studio may include a backdrop 410. The backdrop may be of a colour selected to improve the sensor's ability to obtain accurate measurements. For example, the backdrop may be a green screen, or may be blue or any number of other colours selected to provide contrast between the subject and the backdrop.

A sensor 420 may be provided. The sensor may include one or more sensors of the same or different types. The sensor may be configured to measure motion and/or obtain data, such as dimensions of a body or part, the position of same, and/or motion of same over a period of time. The sensor may include a kinematic data capture device. The sensor may include a camera. The sensor may include a lidar sensor. The sensor may receive data and transmit same to the computer for processing and/or storage. There may be multiple sensors, which may be of the same or different types. The sensor may be disposed to obtain whole body data of a subject, and/or may be disposed to obtain data from a particular part of the body, that is, the sensor may be configured to obtain data of a body segment. For example, various sensors may be positioned at different angles and positions with respect to the subject. Redundant sensors may be used, which may improve reliability of data collected. Complementary sensors may be used to collect data using different methods. For example, a conventional camera and a lidar sensor may be used to collect related data about the same subject, from the same or different positions or perspectives relative to the subject. Similarly, two of the same sensor type, for example two cameras, may be disposed at different positions with respect to the subject.

The sensor may include a mass sensor, which may include an apparatus to obtain mass data of the subject, for example a scale, such as a weight scale, or pressure plate. In one aspect, the mass sensor may be disposed on the surface where the subject is to be positioned during part or all of the subject's assessment. In one embodiment, the sensor may measure movement in one or more degrees of freedom, including one or more of surge (forward/back), heave (up/down), sway (left/right), yaw (normal axis), pitch (transverse axis), and roll (longitudinal axis). Accordingly, the sensors may collect data regarding balance, gait, and athletic, clinical, and occupational motion. Such data and assessments may be recorded in standardized formats such as C3D (www.C3D.org). C3D files are a standard that contain information that may enable review, display, and analysis of 3D motion data, and may include additional analog data from force plates, electromyography, accelerometers, and other sensors.

Motion capture data may be derived and/or collected using any one or more of various sensors, methods and apparatus, for example, full- and/or partial-body methods and apparatus, kinematic tracking methods and apparatus, kinetic tracking methods and apparatus, and/or electromyographic tracking methods and apparatus, naked-eye observation, cameras, optical photo and/or video cameras, inertial sensors, magnetic means, infrared means, accelerometers, gyroscopes, image analysis technology, manual goniometer, static with/without dynamic force plates, markerless image acquisition, marker-based image acquisition, lidar (light detection and ranging), laser scanning, ultrasound imaging, x-ray, computed tomography, magnetic resonant imaging, tomographic imaging of any type, positron emission tomography, nuclear imaging, fluoroscopy, neutrino tomography, photographic imaging of various wavelengths or types, elastography, photoacoustic imaging, infrared spectroscopy, magnetic particle imaging, and optoacoustic imaging, to name a few.

The sensor may sense, observe, and/or detect, one or more forces or events. For example, a first sensor may detect kinematic force and associated data. A second sensor may measure a body segment's dimensions, for example length. A third sensor may measure mass of a body segment. A fourth sensor may detect force being generated by a subject at a particular position and/or in a particular direction. The position and/or direction being detected may be selectable, and in use may be selected by the technician and/or the processor. In one embodiment, wherein the sensor is configured to detect force, the sensor may include a force plate for such purpose. Alternatively, the sensor may detect force without a force plate.

A computer 430 may be provided, which may include a processor to compute, retrieve, store and write data, and execute instructions, such as an algorithm, software, etc. The computer may do, support, perform, and/or carry out one or more of the functions described herein. The computer may include a module, such as a storage medium, coupled to the processor, to store data. The computer may include and/or be coupled to a user interface 432, for example a touch screen, which may display reports and provide a means for a technician to send signals to the computer and receive signals therefrom. The computer may be coupled to the sensor and may receive data therefrom. The computer may control the sensor. The computer may receive data from other sources, for example, from a technician or from the subject.

The computer may store and retrieve raw data, meta-data, and/or processed data, and other forms of data, using one or more data storage formats or standards. Examples of such formats or standards include DICOM (digital imaging and communication in medicine) JPEG, BNP, MPEG, etc., on storage systems such as picture archiving and communication system (PACS), and other medical imaging technologies, which may provide economical storage and convenient access to images from multiple modalities. The storage medium and/or processor may be cloud-based. In one aspect, cloud-based apparatus may provide on-demand availability of computer system resources, such as data storage and processing power, optionally without direct active management by the user.

The computer may receive background data about the subject, for example, the subject's age, sex, height, weight, ethnicity, athletic abilities or desires, medical history, family history, etc. Such information may be collected, for example, via a user interface, for data entry by the subject and/or technician.

The computer may have, create, and/or update comparator data. Data about the subject may be compared to comparator data, by the processor. The processor may extract key image or waveform details, for example, for the purpose of disseminating important information to the back to the original motion waveform data. The image data can be correlated to the waveform data so that features of the waveforms can be correlated to the subject's actual motion images. For example, the shoulder abduction waveform of a subject may be correlated to the actual images used to acquire and calculate said waveform.

The processor may generate a report 434 about the subject, which may include a comparison with comparator data. The processor may receive data from the sensor. The processor may process motion capture data. The processor may transform and/or render motion data into a time domain matrix of three-dimensional vectors (TDM3DV), which may be compared across subjects.

The processor may process and/or reconfigure motion capture data into processed data, which may include waveform data, frequency, anatomical motion, planar motion, measured forces, and/or estimated forces. The processor may correlate waveform data from different data acquisition technologies. The processor may calculate a motion waveform from the motion capture data. The motion waveform may include an average and/or summary of motion of a subject. Further, or in the alternative, the comparator data may be generated over time, as the computer collects data from one or more subjects during one or more sessions or iterations. Collected data may be integrated into the comparator data. The processor may augment comparator data with collected data such that the comparator data includes the collected data, or a portion thereof. Advantageously, such iterative improvements and augmentations to comparator data over time and subjects may make the comparator data and any systems, apparatus, and methods supported thereby more reliable. An apparatus, which may be a set, may be standardized to permit location-agnostic analysis. For example, the relative positions of elements of the set, such as the one or more sensors, backdrop, and a subject position, i.e., where a subject is to be positioned during analysis, may be standardized and the subject may attend a first set for their first session and another set for a subsequent session.

In use, the subject may assume one or more maneuvers. Such maneuvers may be selected according to the body segment being analyzed, and/or according to particular cohorts of interest.

The computer may be pre-programmed with comparator data. Comparator data may pertain to, or describe, average motion within a confidence interval, describing the distribution of motion within a group of subjects in a cohort. A group of subjects with one or more common traits may be referred to as a cohort. The processor may process motion capture data, for example, by generating a waveform, with excursions and peaks, which may be included in the report and/or compared to cohort data. Cohorts may overlap. Put another way, a cohort may be defined by any number of attributes or traits. For example, a cohort of subjects aged 50-60 may overlap with a cohort of individuals with arthritis. Another cohort could be characterized by subjects recovering from whiplash injury who are obese males (BMI 35-40) over 60 years of age. Cohorts may be generated by the processor using a relational database of motion movement associated with different individuals and each of their associated traits. Regression analysis may be used to isolate traits as variables of interest.

Traits may include, for example, one or more of: biophysical characteristics (e.g., body mass index (BMI), whether the subject is healthy, whether the subject has, or is suspected to have, a particular state, condition, ability, injury, disease, or illness, or combination thereof, the subject's exercise history, the subject's treatment history) and/or demographic characteristics (such as age, sex, gender, occupation, ethnicity, etc.), and the temporal progression of such traits with our without treatment, training, or other interventions. Certain diseases or states of interest may include stroke, Parkinson's disease, athletic injuries, motor vehicle accident history, whiplash, and other trauma, to name a few. Time since injury or another incident of interest may be another trait included in the analysis. In one embodiment, such traits may be assessed in order to rule out such traits. For example, regardless of whether the subject has any indications of having had a stroke, the subject's data may be compared to stroke comparator data and a report may be generated directed to same.

The computer may have a normative motion database, against which subjects may be compared to identify similarities and differences, which may be used to inform diagnosis, prognosis, treatment options, cost of care analysis, and other applications. In one aspect, a means to compare motion data from different motion capture systems is provided, which may determine the factors needed to correlate data from such systems to create a standard of normative motion data.

Motion data about a subject may be compared to comparator data of one or more cohorts. In use, data collected about a particular subject may be compared with comparator data to identify differences and/or similarities, which may inform diagnostic, prognostic, and other reports generated by the computer. Artificial intelligence (AI), for example, machine learning, pattern recognition, and/or related methods, may be used. For example, AI may associate data about a subject with a cohort. AI may associate the subject's data with health conditions. The processor may compare a waveform associated with the subject with a waveform associated with a cohort and determine that the subject is part of the cohort. The processor may record the subject's data as part of the cohort's data. The processor may prompt the technician to provide further information, for example, to confirm whether the subject's data should be recorded as part of the cohort's data. The processor may determine that the subject does not fit within a cohort, i.e., the subject's waveform does not correspond to a waveform of a cohort. The processor may create a new cohort. The processor may determine that the subject is part of any number of cohorts. The processor may receive a signal, for example from the technician, indicating a particular cohort of interest, and the processor may cause the report to include information regarding the closeness of the subject's data to the cohort, for example by showing whether the subject's waveform fits within the margin of error of the cohort.

A subject may be assessed multiple times. For example, the subject may be assessed immediately after an injury, and then again after surgery, and again one month after surgery, to measure progress and the body's response to the surgical intervention. In this sense,

The processor may ignore certain cohorts based on information received about the subject. For example, if the processor receives information that the subject is elderly, the processor may ignore youth cohort data. Comparison between the subject's data and that of comparator cohorts may be useful in any number of applications, including to measure wellness and/or athletic training.

The comparator data may include one or more comparator data sets. In one embodiment, a comparator data set may pertain to a cohort, for example, a cohort of subjects with a particular pathology, disease, abnormality, advantage, or trait. For example, a comparator data set may pertain to certain traits about the cohort, for example, that its subjects are marathon runners. Another comparator data set may pertain to subjects with Parkinson's disease, stroke, neurological trauma or disease, musculoskeletal trauma, arthritis, and/or arthralgia, to name a few. Alternatively, comparator data may pertain to subjects that are healthy. Cohorts may be based on any number of factors, including any one or more of: age, sex, athleticism, participation in a specific sport (e.g., one or more of figure skating, gymnastics, long- and short-distance running, swimming, etc.). Information about such factors may be collected from subjects and considered as part of the processor's analysis. Comparator data may be collected from the present system and method or from other data available in the art of the invention.

In one embodiment, the motion waveform may have a confidence interval describing the distribution of motion within a cohort. This waveform and/or confidence intervals may be derived using one or more methods, including: autocorrelation, regression (e.g., techniques based on one or more of: linear least squares, non-linear, ordinary least squares, logistic, stepwise, polynomial, ridge, lasso, elastic net, quantile, partial least squares, poisson, negative binomial, Bayesian linear, corrective, retest-all, selective, progressive, complete, partial, unit, etc.), techniques in time, frequency, spatial or other domains, and/or using transforms (such as Fourier, Laplace, Hankel, Hilbert, Jacobi, Wavelet, Weierstrass, Z-transform, Chebyshev, etc.).

The processor may generate a report 434. The comparison information may be included in the report. For example, the processor may generate a report that indicates that the subject's performance is deteriorating, and therefore a given disease is advancing. This may inform medical interventions, and/or the subject's decision-making, for example whether to continue to pursue sport or other activities that require movement of their body. The report may display data, for example in raw form, in a visualized format (e.g., a waveform), and/or interpretation of data (e.g., the subject may have had a stroke based on a comparison of the subject's data with comparator data of stroke patients). The processor may compute, and/or cause to be included in the report, clinical, athletic, and/or wellness diagnostic and prognostic information, based on the subject's motion waveform. The processor may compute, and/or cause to be included in the report, suggested interventions, for example training, treatment, and/or therapy. Such suggested interventions may be computed based, in part, on the motion capture data and other data about the subject.

The processor may cause the report to be, for example, printed, saved to the storage medium, transmitted to another device, and/or displayed on the user interface 432, such as a screen.

Advancements in markerless motion image capture technology has reduced the amount of time taken to acquire motion data, and has improved accuracy and repeatability, thereby improving such technologies suitability for certain applications, e.g., athletic or wellness evaluations, such as diagnosis of and assessing recovery from a stroke. Other applications for motion capture technology include augmented reality for gaming and the like.

The multitude of motion capture technologies which enable real-time and/or just-in-time analysis of motion and locomotion are becoming ubiquitous. Such technologies may support methods to compare a subject against themselves, or another individual. However, the means to compare them against a similar cohort of individuals or a population of interest (for example, for diagnosis or training) is wanting. A database of normative motion of whole-body and/or body segment motions is needed to compare a specific movement (e.g., hip abduction) performed by specific subject or patient cohort (e.g., male age 35-45, body mass index 30-40). This can allow the subject to understand their performance compared to themselves or a matched cohort. It may also allow the patient, subject, and subject matter expert (e.g., physician, trainer, and/or therapist) to evaluate training and/or therapeutic interventions on the subject's trajectory towards the normative or desired motions of a matched cohort.

For example, the sensors may collect motion capture data about a motion (such as shoulder abduction) of a subject, and the processor may receive such data and compare same with a cohort of interest (for example, a cohort defined by the traits of individuals who are female, aged 20-30, having a BMI between 20-25). A normative waveform may be generated for the cohort, with confidence intervals. Such waveforms may be generated using temporal and/or spectral comparison and/or correlation methods. With reference to FIG. 5, shoulder abduction waveforms 52, 53, 54 may be temporally and spectrally auto-correlated to derive the normative waveform 50. With reference to waveform 60 of FIG. 6, in testing, it was observed that 50 subjects can be used to create a cohort with 95% confidence intervals using the present methods and apparatus.

With reference to FIG. 1 and FIG. 2, various methods and apparatus herein described may be used to acquire and process motion data. Data about movements of various characteristics and complexities may be assessed. For example, motion can be a single movement (see FIG. 1), or a repetitive motion such as walking (see FIG. 2). Motion of an entire body may be assessed, or the assessment may be directed to a part of the body, such as a joint or limb. Multiple subjects may have the motion of a particular limb analyzed. For example, FIG. 1 illustrates example data of a shoulder abduction, and FIG. 2 illustrates example data of knee flexion during walking.

With reference to panel A of each of FIG. 1 and FIG. 2, joint angle movement data of n subjects are plotted as a function of time. The time domain plot can include joint angle, limb movement, and/or motion of any point of the body such as the centre of mass. In other words, the horizontal axis may indicate time, and the vertical axis may indicate one or more of joint angle, limb movement distance, and/or motion data (such as distance moved) of any point of the body, such as centre of mass.

Each individual subject's motion may be scaled to a fixed number of time samples using spectral scaling and/or filtering, as illustrated in panel B of each of FIG. 1 and FIG. 2. The scaling may be performed in a manner that does not compress or expand time. The computer may spectrally scale motion capture data to a common sample rate. The computer may scale, compress or otherwise alter the data in a manner which does not substantially degrade the data, i.e., in a lossless manner. Alternatively, the computer may scale, compress or otherwise alter the data in a manner that degrades the data within a tolerable limit, which may be selected by the technician.

The waveforms can then be compared with each other by using temporal and/or spectral autocorrelation techniques, for example, to minimize the root-mean-squared error and/or least-means-squared error of a chosen cohort of subjects. Each individual subject's processed motion information can be compared to the average waveform of processed motion information about the whole cohort. A maximum error may be established to exclude those individual subject waveforms which are not within a range of average waveforms or confidence intervals for waveforms of the whole cohort (see, e.g., panel C of each of FIGS. 1 and 2). This can result in a multitude of combinations (e.g., n*(n+1)/2) of all waveforms to derive a master cohort waveform which best represents the cohort, optionally after the exclusion of those waveforms which do not fit within the cohort (see, e.g., waveform Z of panel D of each of FIGS. 1 and 2). An error or variance around the waveform (see, e.g., waveforms X of panel D of each of FIGS. 1 and 2), which may be considered an upper and/or lower bound, can be used to derive a measure of the fit of waveforms in the cohort (see, e.g., panel A of each of FIGS. 1 and 2) to the master waveform (see, e.g., panel D of each of FIGS. 1 and 2).

For any given motion of a body, there can be several comparator waveforms, and confidence intervals, across different cohorts. For example, waveforms and confidence intervals for a shoulder abduction movement may be different in a cohort of healthy individuals than in a cohort of injured individuals (for example, individuals with a complete infraspinatus muscle tear).

Using comparator data of one or more cohorts, which may comprise motion data of the same movement but across different populations and/or pathologies, a subject can be assessed and compared to such cohorts, to determine which of the cohorts the subject may be a part. Such a method may help identify illnesses or pathologies.

With reference to panel D of FIG. 3, the waveforms X, Z could represent a cohort of individuals who have suffered the same injury, or a healthy cohort of normal joints. A new subject (see, e.g., FIG. 3, panel E) could have their waveform time corrected and/or scaled (see, e.g., FIG. 3, Panel F), in a manner similar to all the waveforms in the cohort being used for a comparison (see, e.g., FIGS. 1 and 2, panel B, and FIG. 3, Panel D) and then compared.

Error values or estimates may be calculated to assist in determining the “fit” of the subject into the cohorts. In other words, such error figures may assist in determining the comparator cohorts, if any, to which the subject may bear resemblance or belong.

For example, a subject with a complete rupture of their infraspinatus muscle in their rotator cuff may exhibit motion data with a waveform that fits the cohort comparator waveform for this injury. As the subject progresses, possibly in recovery supported by treatment (surgical or otherwise), their waveform may begin to show increased error from the injured cohort and less error towards the healthy cohort. In this aspect, the present apparatus and method may measure or track outcomes, for example, progress, healing, injury status, permanence of injury, and success of interventions. The processor may calculate and/or display such information in the report. In one embodiment, this may be illustrated using parametric and graphical data of this subject's motion data changes over time, to represent a permanent injury or an objective measurement of progress towards the healthy cohort (see, e.g., FIG. 3, panel F).

In one embodiment, in use, the system may function substantially automatically. For example, a subject may position themselves within the area detectable by one or more sensors. The computer may be initiated and instructed to begin collecting data and performing analysis. The subject may make a movement for analysis. The computer may be instructed to stop capturing data. The computer may generate a report. The computer may cause the report to be displayed. In one embodiment, the computer may conduct motion capture analysis without human input except to activate the computer and software, start and stop capturing data, and, optionally, instruct the subject regarding the motion to be performed, which may be selected according to a specific cohort or question being posited for analysis.

In one embodiment, the computer may display or otherwise communicate (for example, audibly via speakers) directions for the subject. Such directions may include, for example, to move forward or backward, to perform certain bodily movements, to move closer or further away from the sensor, etc. This may be done with or without technician supervision. Such directions being communicated by the computer may increase repeatability and consistency between subjects and sessions, and reduce human error.

The centre of mass (COM) is a point that may be used in analysis of different human body configurations, both under static and dynamic conditions. To maintain balance, which a subject generally needs or desires during static conditions, the subject may move such that they keep the projection of their centre of mass above an area of equilibrium. Centre of mass helps in drawing an angle of equilibrium. During a movement, the centre of mass can be used as a point which substitutes the whole body in description of sinusoidal locomotion of the human body in vertical and horizontal planes. The processor may determine the subject's centre of mass, including by using sensor data.

Because the COM of a body is dependent on the distribution of its mass, the COM location for a rigid body (e.g., one that does not experience any substantial change in shape) will be substantially fixed. In contrast, the COM of a body whose mass distribution can change (e.g., the human body) will not necessarily have a substantially fixed location, that is, the body's COM location may be unfixed. In addition, it is important to keep in mind that the COM location may sometimes fall outside of the body. A doughnut, for example, has its COM in the “hole” in or near its middle.

Masses and locations of centres of mass of body parts may be measured. For example, one or both of following two methods may be used to determine a location of a centre of mass of a whole body: (a) sum of masses; and (b) sum of moments.

In the sum of masses method, the computer may receive mass and distance data, and determine the centre of mass location by adding the masses and distance between them. For example, a common centre of mass of foot and shank can be obtained by connecting centres of mass of foot and shank. The common centre of mass will lie on a line connecting these two centres of mass. By adding the masses, the computer determines information on a location of common centre of mass. Using this approach, one can obtain further common centres of mass up to the centre of mass of the whole body.

In the sum of moments method, a reference system is used, drawn near the centres of mass of two body parts. Distances are measured between centres of mass and the reference system. Masses are multiplied by distances separately for both axes. Thus, moments of mass are obtained. These moments are summed, and the result is divided by sum of masses. This gives a distance of common centre of body parts from the reference system. For the whole-body COM, one may make a projection onto the reference system of all centres of mass of body parts. In a computer-implemented method, instead of centres of mass, a position of joints may be given for analysis by the computer program.

In the case of the human body, COM may be dependent on body habitus, since the distribution of body segment masses can be quite different between any two individuals. Nonetheless, using body segment analysis based on markerless motion analysis makes this COM calculation possible using any number of COM algorithms. If the body composition of an individual is constant over time, then the COM should be fixed in place and other methods of body motion analysis (multiple cameras, LiDAR etc.) could be used to follow the COM and balance of a subject over time.

Markerless body motion analysis can also measure dynamic balance stability. The motion of the segments of the body during standing and certain maneuvers can reveal a pattern of movement, and classify such patterns as normal or abnormal, e.g., pathological. In many cases, the pathology will cause the subject to have a certain pattern of movements unique to their illness or injury and the history of its progression, for example, deterioration, or recovery. Using artificial intelligence and/or algorithms, which may be implemented by a computer, these patterns can be considered for the purposes of diagnosis, prognosis, and projected or real response to treatment, among other applications.

For example, a patient suffering from concussion may have large deviations of their COM relative to their centre of gravity before taking corrective action to maintain posture and stability. As the patient recovers, the deviations may decrease, form may improve (i.e., tend to the norm), and/or corrective actions may be more frequent or consistent. Patients with motor weakness or disability, such as from trauma or stroke, may need to adapt their posture to favor the stronger and healthier components of their musculoskeletal system. As the subject heals, these patterns may slowly resolve to normal. Patients with arthritis may favour loading their skeleton in a manner to reduce pain and maintain balance and posture. Provocative maneuvers such as squatting and jumping may reveal significant pathology which could identify certain subjects as unable to perform essential tasks in their activities of daily living, work environment or driving a motor vehicle.

An index of a subject's balance and posture, which may include the body's response to maneuvers, may be created using data relating to dynamic measurements such as the subject's motion over time, their static postural stability, and/or the body's response to maneuvers. Such an index could inform an assessment of the severity of a non-dominant stroke and its estimated time course of recovery, or whether the subject may be permanently impaired. These findings would help the patient subject and their care provider prognosticate on recovery for the purposes of planning for future support, and possible insurance implications, among other applications. Although many strokes can occur in a similar anatomical area of the brain, the recovery can be quite variable depending on the diagnosis (e.g., thrombotic or hemorrhagic), initial treatment and underlying pre-existing disease (e.g., atherosclerosis, diabetes, hypertension, etc.). If a normative index of balance is established which can be used to compare the patient's initial presentation and their subsequent progression, a metric of functional loss can be objectively determined for prognosis and actuarial (e.g., insurance, cost of care analysis, etc.) uses, among other applications. This normative index of balance can also be used to help optimize performance of athletes for training and recovery from injury. The balance index can be categorized by gender, BMI, age, sport, etc., so subjects can judge their performance relative to a matched cohort, and trainers can adjust exercise and techniques.

COM from sacral point on BVH has been shown to be comparable to COM calculation for relative COM between studies. However, any other reference systems that match joint position in space and time may be used instead or in addition.

Hip joint centre relative to COM and/or other joints may be measured. Knee joint centre relative to COM and/or other joints may be measured. Ankle joint centre relative to COM and/or other joints may be measured, and can also be used to measure point of contact with ground to determine centre of gravity and ground reactive forces relative to COM.

Sway/area example: with reference to FIG. 5, sway/area−how far COM (1) or (2, 3, 4) move.

Response to perturbation example: how quickly/efficiently recovery is accomplished or decompensation occurs.

Coordination control example: how the body system intercedes to adjust to perturbation. Reconstructed coordinate information may be obtained from motion capture for relative joint and centre of mass positions. This may be obtained in either 2D or 3D Cartesian or polar coordinate systems. Coordinate data in X, Y and/or Z directions may be obtained. Coordinate data may be plotted and/or converted into a waveform, e.g., as a function of time. Area/sway as a function of time may be calculated. This may be, for example, as a total or at discrete periods of time. Each plane (X, Y, Z) may be used. For each plane (X, Y, Z), the waveform may have a frequency. Area/sway and frequency profiles may be used to generate a normative or pathological cohort data set. Analysis, which may be characterized as scoring, against a particular cohort, may be used to create a sway score and/or a frequency score.

Scores can be derived mathematically, e.g., by the processor, by comparing the subject's motion waveform or COM waveform, or range of motion or any other motion characteristic, to a comparator waveform for that particular movement or motion. The comparisons used to derive a score, for example, a measurement of difference or similarity, which can be computed using any number of analytical or statistical methods, such as autocorrelation, regression (e.g., techniques based on one or more of: linear least squares, non-linear, ordinary least squares, logistic, stepwise, polynomial, ridge, lasso, elastic net, quantile, partial least squares, poisson, negative binomial, Bayesian linear, corrective, retest-all, selective, progressive, complete, partial, unit, etc.), techniques in time, frequency, spatial or other domains, and/or using transforms (such as Fourier, Laplace, Hankel, Hilbert, Jacobi, Wavelet, Weierstrass, Z-transform, Chebyshev, etc.). Two or more waveforms, which may be planar waveforms, may be compared with each other, for example mathematically, statistically or otherwise, e.g., using circular statistical methods or parametric mapping. Such comparisons may be included in the report.

These statistical methods may be applied to waveforms that may have been interpolated to have substantially the same number of samples. This temporal scaling allows the shape of the waveforms to be compared using autocorrelation techniques, among other methods stated above, however, spectral distortion could occur. To address possible spectral distortion, other methods that do not use waveform interpolation may be applied to facilitate the comparison of a subject with the spectra or phase of comparator data of a cohort.

Either way these waveforms are analyzed the resulting magnitude of variance or deviation between an individual waveform and a similar cohort is converted into a score depending on the pathology of circumstance surrounding the subject in question. Sometime these scores are calculated using weighting factors from clinical and other subject matter experts.

In one embodiment, the method includes a subject testing and/or data collection procedure. A subject may present for assessment, for example at a studio. The studio may have one or more sensors disposed at fixed and/or mobile locations, which may be coupled to the computer.

Prior to, or during, the assessment, biographical data about the subject may be collected, for example, using a user interface associated with a computer. Biographical data may include name, date of birth, address, email, telephone number, referring physician or other medical professional, other advisors, such as personal trainers, physiotherapists, etc., and/or other information about the subject. Health information, such as medical history, height, weight, sex, musculoskeletal injury history, pathological/disease history and pharmacological history may be obtained, for example, directly from the subject, through a technician- and/or computer-assisted interview or survey, from a medical professional, etc. Details regarding anatomical location, relevant medical investigations, outcomes, surgical procedures and date of injuries may be collected.

Legal consents, terms, and conditions may be presented, negotiated, completed, and/or accepted by, for, or on behalf of the subject, for example using the user interface. For example, consent may be obtained to use the subject's non-identifiable, anonymous, and/or de-attributed data, optionally including health information and motion capture information, for research purposes and/or for integration into comparator data. Optionally, the subject may be able to withdraw their consent, or provided with instructions regarding how to do so.

The subject may change into clothing, optionally including shoes, selected for their size and the segment(s) of their body being analyzed. Clients may be encouraged to use the same clothing for subsequent assessments. Alternatively, the subject may be prompted not to wear footwear. A set standardized testing assessment protocol may be selected, for example, by the subject and/or a medical professional.

The assessment procedure and data capture may be conducted by trained technicians, or autonomously or semi-autonomously by the computer. Tests may include various positions or movements. For example, the subject may raise their hands above their head, squat as far as possible to the floor, be given specific start and end positions, etc. The computer and/or technician may observe the subject, e.g., for failure parameter guidelines. Optionally, subjects and/or technicians may be presented with an opportunity to review individual test procedures, and decide whether they wish to skip one or more procedures, or add new ones for analysis.

Sensors, which may be configured for motion capture and human motion measurement, may be disposed in the studio at selected locations and/or distances relative to a subject spot or area, wherein the subject moves and is assessed. Motion capture/measurement devices may be fixed in location using a central point of origin or reference, which may also be positioned a selected vertical distance from a surface such as a floor. A given sensor's distance to the central point may be calibrated using a measurement device (e.g., a laser measurement device). Orientation and direction of the sensor(s) may be zeroed to the central pint of origin using a visible laser guidance tool, which may be performed by the computer. The sensor may be mobile, for example, may travel along a track during an assessment, or may be moved to a particular position depending on the segment of the body being assessed, or according to the subject's dimensions. Information about the positions and orientations of the sensors may be associated with any collected data, and such variables may be controlled and/or adjusted when being compared against comparator data or historical data of the subject. The sensor(s) may be calibrated such that they act substantially synchronously with each other and/or the computer, and in accordance with manufacturer, technical, or other specifications.

A floor and/or background of the studios, such as the backdrop, can be disposed in natural surroundings and/or in enhanced environments. Motion labs may be enhanced studio environments, for example with standardized floor and background colours, acoustic properties and/or lighting. The studio may have a floor decal to show the spot or area of the motion capture studio where the subject should be positioned. A user interface, such as a screen, may be positioned within the subject's field of view, proximate the floor decal, for communication with the subject.

During the assessment, the subject may be instructed to stand on the decal or at another location in the studio. The subject may be instructed to assume a position, for example, the pose of Leonardo da Vinci's Vitruvian Man. This pose may allow for repeatable measurement and anthropometric scaling, which may be used to create a digital three-dimensional (3D) anatomical model of the subject. Assumptions of joint positions and orientations can be made based upon this model. A real-time or post-processed 3D model may be rendered by the computer, e.g., using software, and the subject's joint and/or limb motion may be tracked in 3D space, e.g., expressed as polar or Cartesian coordinates recorded over time.

The subject's adherence to the instructions may be assessed and corrective instructions may be provided, for example to reposition the subject's feet or other body parts. This may be repeated until the subject substantially assumes the given pose and/or performs the given movement. Actual poses and movements may be recorded and used as guidelines for future sessions, e.g., for repeatability purposes.

As the subject moves, the coordinates of individual limb segments may be captured as coordinates at periods. Such periods may be informed and/or defined by a sample rate of the motion capture measurement device, which may also be selected.

The Nyquist Theorem states that, in order to adequately reproduce a signal, it should be periodically sampled at a rate that is twice the highest frequency recorded. In the study of human motion, sampling rates often range, for example, from 60 Hz to 2000 Hz.

Using geometric and mathematical principles, angles between two body segments can be measured and used to infer angles between one of such segments with a third segment. Using time, angular velocities and accelerations can be derived. Examples of such principles are illustrated by the diagram and equation shown in FIG. 7.

Angular velocity, acceleration, and position of joints, over time, may be measured and visualized as a waveform.

By further obtaining weight, e.g., using a pressure plate sensor, and deriving mass, optionally with other anthropometric data (e.g., limb length, limb volume, height, posture, body mass index, circumferences of limbs, chest, or head, etc.), the estimated mass and centre of mass position of individual limbs can be calculated. For example, this can be derived using Dempster's 1955 model, which guides the approximated position of COM in each limb. For example, if the forearm is 10 inches (25.4 cm) long, and the centre of gravity is found 40% from the proximal end, then the COM of the forearm is a distance of 4 inches (10.2 cm) from the proximal end of the forearm.

The entire body's centre of mass can be estimated by determining the centre of mass location of each segment of the body (e.g., as an X, Y pair of coordinates). For example, one or more the following operations may be implemented:
(a) Calculating the torque about a reference point due to each segment (based on the segment's mass and position);
(b) Summing the torques about the reference point for all the segments (a first sum for X direction, a second sum for Y direction);
(c) Dividing the sums of the torques by the total body mass to determine the centre of mass location with respect to the reference point.

The torque about the reference point due to the whole body is equal to the sum of the torques about the reference point due to the body segments. This principle can be characterized by the equation:

$${X}_{cm} = \frac{{\sum }_{i = 1}^{n} ( {M}_{seg i} * {X}_{seg cm i} )}{{M}_{body}}$$

Relative COM position can also be estimated by tracking the sacrum in 3D space. Generally, this is not done as an absolute position. This method for calculating and tracking COM allows further derivative information to be obtained, such as vertical ground reaction force estimated using the second derivative of COM vertical displacement. It is to be understood that the foregoing requires fewer processing steps than methods currently available, thereby improving the efficiency, and reducing the processing power required, of the computer carrying out these functions.

It is to be understood that although human subjects are used herein as an example, motion of other subjects, such as animal subjects, e.g., dogs, horses, etc., can be analyzed using the methods and apparatus described herein.

### CLAUSES

Clause 1. A system for analyzing data from motion capture measurements, comprising: a sensor to measure motion capture measurements; a module configured to receive sensor data from the sensors; and a processor configured to analyze to the sensor data received from the sensors and create a report of such analysis.

Clause 2. The system of any one or more of the clauses, wherein the processor is further configured to compare the sensor data with a cohort data set and generate a score of fit with the cohort data set, and the report further includes the score.

Clause 3. The system of any one or more of the clauses, wherein the sensor is configured to measure centre of mass data of a subject in motion relative to a fixed point of the environment, and the processor is further configured to analyze the centre of mass data for an indicator of balance of the subject, and the report further includes the indicator.

Clause 4. The system of any one or more of the clauses, wherein the processor is further configured to sort the sensor data into at least one cohort.

Clause 5. The system of any one or more of the clauses, further comprising a user interface configured to display the results.

Clause 6. The system of any one or more of the clauses, wherein the sensor includes a camera.

Clause 7. The system of any one or more of the clauses, wherein the sensor includes a pressure plate.

Clause 8. The system of any one or more of the clauses, wherein the processor controls the sensor.

Clause 9. The system of any one or more of the clauses, wherein the module is further configured to receive background data about a subject.

Clause 10. The system of any one or more of the clauses, wherein the module has comparator data.

Clause 11. The system of any one or more of the clauses, wherein the processor is configured to compare the sensor data to the comparator data.

Clause 12. The system of any one or more of the clauses, further comprising a backdrop.

Clause 13. The system of any one or more of the clauses, wherein the sensor includes a stationary sensor.

Clause 14. The system of any one or more of the clauses, wherein the sensor includes a mobile sensor.

Clause 15. A computer-implemented method of analyzing data from motion capture measurements, comprising: performing motion capture measurements on a subject; receiving motion capture data from the motion capture measurements; processing the motion capture data and the background data on a computer using an algorithm, including comparing the motion capture data and background data to one or more comparator waveforms; generating a report based on the processing analysis; and displaying the results of the report.

Clause 16. The method of any one or more of the clauses, further comprising receiving background data about the subject.

Clause 17. The method of any one or more of the clauses, further comprising storing the motion capture data and background data to a historical data file for the subject.

Clause 18. The method of any one or more of the clauses, further comprising comparing the motion capture data and background data to a historical data file of the subject.

The previous description of the disclosed embodiments is provided to enable any person skilled in the art to make or use the present invention. Various modifications to those embodiments will be readily apparent to those skilled in the art, and the generic principles defined herein may be applied to other embodiments without departing from the spirit or scope of the invention. Thus, the present invention is not intended to be limited to the embodiments shown herein, but is to be accorded the full scope consistent with the claims, wherein reference to an element in the singular, such as by use of the article “a” or “an” is not intended to mean “one and only one” unless specifically so stated, but rather “one or more”. All structural and functional equivalents to the elements of the various embodiments described throughout the disclosure that are known or later come to be known to those of ordinary skill in the art are intended to be encompassed by the elements of the claims. Moreover, nothing disclosed herein is intended to be dedicated to the public regardless of whether such disclosure is explicitly recited in the claims. No claim element is to be construed under the provisions of 35 USC 112, sixth paragraph, unless the element is expressly recited using the phrase “means for” or “step for”.

1. A system for analyzing data from motion capture measurements, comprising:
a sensor to measure motion capture measurements;
a module configured to receive sensor data from the sensors; and
a processor configured to analyze to the sensor data received from the sensors and create a report of such analysis.

2. The system of claim 1, wherein the processor is further configured to compare the sensor data with a cohort data set and generate a score of fit with the cohort data set, and the report further includes the score.

3. The system of claim 1, wherein the sensor is configured to measure centre of mass data of a subject in motion relative to a fixed point of the environment, and the processor is further configured to analyze the centre of mass data for an indicator of balance of the subject, and the report further includes the indicator.

4. The system of claim 1, wherein the processor is further configured to sort the sensor data into at least one cohort.

5. The system of claim 1, further comprising a user interface configured to display the results.

6. The system of claim 1, wherein the sensor includes a camera.

7. The system of claim 1, wherein the sensor includes a pressure plate.

8. The system of claim 1, wherein the processor controls the sensor.

9. The system of claim 1, wherein the module is further configured to receive background data about a subject.

10. The system of claim 1, wherein the module has comparator data.

11. The system of claim 1, wherein the processor is configured to compare the sensor data to the comparator data.

12. The system of claim 1, further comprising a backdrop.

13. The system of claim 1, wherein the sensor includes a stationary sensor.

14. The system of claim 1, wherein the sensor includes a mobile sensor.

15. A computer-implemented method of analyzing data from motion capture measurements, comprising:
performing motion capture measurements on a subject;
receiving motion capture data from the motion capture measurements;
processing the motion capture data and the background data on a computer using an algorithm, including
comparing the motion capture data and background data to one or more comparator waveforms;
generating a report based on the processing analysis; and
communicating the results of the report.

16. The method of claim 15, further comprising receiving background data about the subject.

17. The method of claim 15, further comprising storing the motion capture data and background data to a historical data file for the subject.

18. The method of claim 15, further comprising comparing the motion capture data and background data to a historical data file of the subject.
